(19) United States
(12) Patent Application Publication
(10) Pub. No.: US 2024/0153646 A1
(43) Pub. Date: May 9, 2024

(54) METHOD, SYSTEM, AND NON-TRANSITORY COMPUTER-READABLE RECORDING MEDIUM FOR MANAGING OUTPUT DATA OF BIOSIGNAL ANALYSIS MODEL

(71) Applicant: HUINNO, CO., LTD., Seoul (KR)

(72) Inventors: Jin Guk KIM, Seoul (KR); Jae Seong JANG, Seoul (KR)

(21) Appl. No.: 18/412,934

(22) Filed: Jan. 15, 2024

Related U.S. Application Data
(63) Continuation of application No. PCT/KR2022/009926, filed on Jul. 8, 2022.

(30) Foreign Application Priority Data
Jul. 16, 2021 (KR) 10-2021-0093828
Aug. 2, 2021 (KR) 10-2021-0101667

(51) Int. Cl. G16H 50/70; G16H 40/67; G16H 50/20

(52) U.S. Cl. CPC G16H 50/70; G16H 40/67; G16H 50/20

(57) ABSTRACT

A method for managing output data of a biosignal analysis model includes the steps of: acquiring analysis result data for a plurality of pieces of biosignal data from a biosignal analysis model; performing clustering on a plurality of pieces of first-type biosignal data analyzed as corresponding to a first type, among the plurality of pieces of biosignal data, on the basis of the analysis result data, and extracting at least one piece of sample biosignal data from at least one cluster generated by the clustering; and reperforming the clustering with reference to feedback on whether the analysis result data for the at least one piece of sample biosignal data is accurate.

### CROSS-REFERENCE TO RELATED APPLICATION(S)

This application is a Continuation of International Application No. PCT/KR2022/009926 filed on Jul. 8, 2022, which claims priority to Korean Patent Application No. 10-2021-0093828 filed on Jul. 16, 2021 and Korean Patent Application No. 10-2021-0101667 filed on Aug. 2, 2021. The aforementioned applications are incorporated herein by reference in their entireties.

### TECHNICAL FIELD

The present invention relates to a method, system, and non-transitory computer-readable recording medium for managing output data of a biosignal analysis model.

### RELATED ART

Due to recent rapid progress in science and technology, the quality of life of all mankind is being enhanced and medical environment has changed a great deal. Particularly, in recent years, wearable monitoring devices that can analyze biosignals during daily life without visiting a hospital have become widely available to the public.

These wearable monitoring devices are provided with a biosignal analysis model to analyze a biosignal of a subject, which is often implemented on the basis of artificial intelligence technology to improve the accuracy of the analysis. Despite the increasing accuracy of the analysis, the analysis results of the biosignal analysis model should be examined by medical personnel (e.g., doctors) in the hospital in order to make a final determination or diagnosis.

The biosignal data measured over days or weeks and the analysis result data of the analysis model for the biosignal data are huge, reaching tens of thousands of pieces, which causes a problem that it takes a lot of time and effort for the medical personnel to examine the analysis results of the analysis model in detail.

### SUMMARY

One object of the present invention is to solve all the above-described problems.

Another object of the invention is to enable medical personnel to efficiently examine analysis results outputted from a biosignal analysis model, without having to examine all biosignal data, by acquiring analysis result data for a plurality of pieces of biosignal data from a biosignal analysis model, performing clustering on a plurality of pieces of first-type biosignal data analyzed as corresponding to a first type, among the plurality of pieces of biosignal data, extracting at least one piece of sample biosignal data from at least one cluster generated by the clustering, and reperforming the clustering with reference to feedback on whether the analysis result data for the at least one piece of sample biosignal data is accurate.

Yet another object of the invention is to effectively increase the accuracy and reliability of analysis result data that is outputted from a biosignal analysis model implemented on the basis of artificial intelligence technology and provided to medical personnel.

The representative configurations of the invention to achieve the above objects are described below.

According to one aspect of the invention, there is provided a method for managing output data of a biosignal analysis model, comprising the steps of: acquiring analysis result data for a plurality of pieces of biosignal data from a biosignal analysis model; performing clustering on a plurality of pieces of first-type biosignal data analyzed as corresponding to a first type, among the plurality of pieces of biosignal data, on the basis of the analysis result data, and extracting at least one piece of sample biosignal data from at least one cluster generated by the clustering; and reperforming the clustering with reference to feedback on whether the analysis result data for the at least one piece of sample biosignal data is accurate.

According to another aspect of the invention, there is provided a system for managing output data of a biosignal analysis model, comprising: a data acquisition unit configured to acquire analysis result data for a plurality of pieces of biosignal data from a biosignal analysis model; and a clustering management unit configured to perform clustering on a plurality of pieces of first-type biosignal data analyzed as corresponding to a first type, among the plurality of pieces of biosignal data, on the basis of the analysis result data, and extract at least one piece of sample biosignal data from at least one cluster generated by the clustering, and to reperform the clustering with reference to feedback on whether the analysis result data for the at least one piece of sample biosignal data is accurate.

In addition, there are further provided other methods and systems to implement the invention, as well as non-transitory computer-readable recording media having stored thereon computer programs for executing the methods.

According to the invention, it is possible to enable medical personnel to efficiently examine analysis results outputted from a biosignal analysis model, without having to examine all biosignal data.

According to the invention, it is possible to effectively increase the accuracy and reliability of analysis result data that is outputted from a biosignal analysis model implemented on the basis of artificial intelligence technology and provided to medical personnel.

### BRIEF DESCRIPTION OF THE DRAWINGS

FIG. 1 schematically shows the configuration of an entire system according to one embodiment of the invention.

FIG. 2 illustratively shows the internal configuration of a biosignal analysis system according to one embodiment of the invention.

FIG. 3 illustratively shows how to cluster biosignal data according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual elements within each of the disclosed embodiments may also be modified without departing from the spirit and scope of the invention. Therefore, the following detailed description is not to be taken in a limiting sense, and the scope of the invention, if properly described, is limited only by the appended claims together with all equivalents thereof. In the drawings, like reference numerals refer to the same or similar functions throughout the several views.

Hereinafter, preferred embodiments of the invention will be described in detail with reference to the accompanying drawings to enable those skilled in the art to easily implement the invention.

### Configuration of the Entire System

Hereinafter, a preferred embodiment of a biosignal analysis system according to the invention will be discussed in detail.

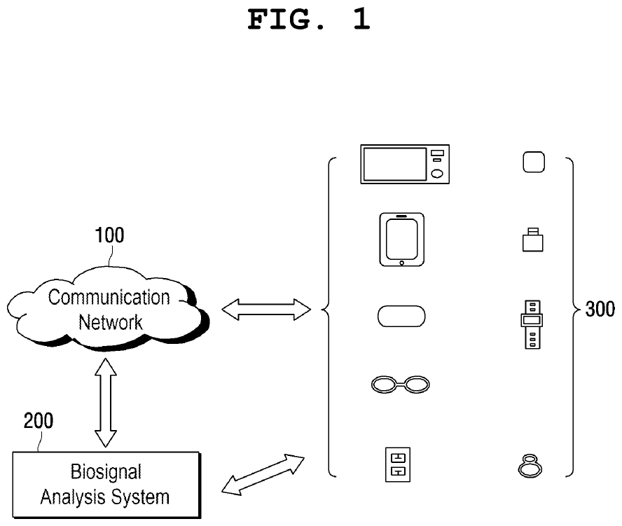

FIG. 1 schematically shows the configuration of the entire system according to one embodiment of the invention.

As shown in FIG. 1, the entire system according to one embodiment of the invention may comprise a communication network 100, a biosignal analysis system 200, and a device 300.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may include known short-range wireless communication networks such as Wi-Fi, Wi-Fi Direct, LTE Direct, and Bluetooth. However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, Bluetooth communication (including Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Next, the biosignal analysis system 200 according to one embodiment of the invention may function to enable medical personnel to efficiently examine analysis results outputted from a biosignal analysis model, without having to examine all biosignal data, by acquiring analysis result data for a plurality of pieces of biosignal data from a biosignal analysis model, performing clustering on a plurality of pieces of first-type biosignal data analyzed as corresponding to a first type, among the plurality of pieces of biosignal data, extracting at least one piece of sample biosignal data from at least one cluster generated by the clustering, and reperforming the clustering with reference to feedback on whether the analysis result data for the at least one piece of sample biosignal data is accurate.

According to one embodiment of the invention, the biosignal analysis model may be a model that outputs a determination result regarding whether analyzed biosignal data corresponds to arrhythmia, or a model that outputs a determination result regarding what type of arrhythmia the analyzed biosignal data corresponds to. For example, the biosignal analysis model according to one embodiment of the invention may be implemented with a binary classification-based artificial neural network where two types (or classes) are normal and abnormal.

The functions of the biosignal analysis system 200 will be discussed in more detail below. Meanwhile, the above description is illustrative although the biosignal analysis system 200 has been described as above, and it is apparent to those skilled in the art that at least some of the functions or components required for the biosignal analysis system 200 may be implemented or included in the device 300, as necessary.

Lastly, the device 300 according to one embodiment of the invention is digital equipment that may function to connect to and then communicate with the biosignal analysis system 200, and any type of digital equipment having a memory means and a microprocessor for computing capabilities may be adopted as the device 300 according to the invention. The device 300 may be a wearable device such as smart glasses, a smart watch, a smart patch, a smart band, a smart ring, and a smart necklace, or may be a somewhat traditional device such as a smart phone, a smart pad, a desktop computer, a notebook computer, a workstation, a personal digital assistant (PDA), a web pad, and a mobile phone.

According to one embodiment of the invention, the device 300 may include a sensing means (e.g., a contact electrode or an imaging device) for acquiring a biosignal from a human body, and a display means for providing a user with a variety of information on the measurement of the biosignal.

In addition, according to one embodiment of the invention, the device 300 may further include an application program for performing the functions according to the invention. The application may reside in the device 300 in the form of a program module. The nature of the program module may be generally similar to those of a data acquisition unit 210, a clustering management unit 220, a communication unit 230, and a control unit 240 of the biosignal analysis system 200 to be described below. Here, at least a part of the application may be replaced with a hardware or firmware device that may perform substantially equal or equivalent functions, as necessary.

### Configuration of the Biosignal Analysis System

Hereinafter, the internal configuration of the biosignal analysis system 200 crucial for implementing the invention and the functions of the respective components thereof will be discussed.

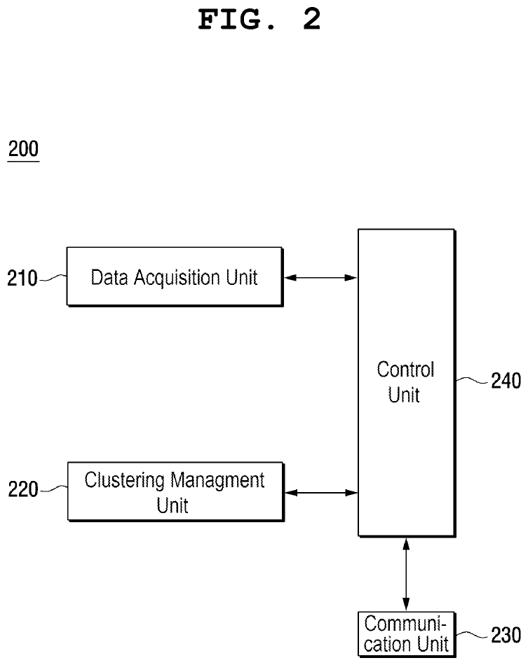

FIG. 2 illustratively shows the internal configuration of the biosignal analysis system according to one embodiment of the invention.

Referring to FIG. 2, the biosignal analysis system 200 according to one embodiment of the invention may comprise a data acquisition unit 210, a clustering management unit 220, a communication unit 230, and a control unit 240. According to one embodiment of the invention, at least some of the data acquisition unit 210, the clustering management unit 220, the communication unit 230, and the control unit 240 of the biosignal analysis system 200 may be program modules to communicate with an external system (not shown). The program modules may be included in the biosignal analysis system 200 in the form of operating systems, application program modules, and other program modules, while they may be physically stored in a variety of commonly known storage devices. Further, the program modules may also be stored in a remote storage device that may communicate with the biosignal analysis system 200. Meanwhile, such program modules may include, but are not limited to, routines, subroutines, programs, objects, components, data structures and the like for performing specific tasks or executing specific abstract data types as will be described below in accordance with the invention.

First, the data acquisition unit 210 according to one embodiment of the invention may function to acquire analysis result data for a plurality of pieces of biosignal data from a biosignal analysis model. According to one embodiment of the invention, the biosignal analysis model may be a model that outputs an analysis result regarding whether the analyzed biosignal data corresponds to arrhythmia, or an analysis result regarding what type of arrhythmia the analyzed biosignal data corresponds to.

For example, according to one embodiment of the invention, the biosignal analysis model may analyze biosignal data of a subject using a machine learning algorithm (e.g., an artificial neural network) to calculate a score regarding whether the biosignal data corresponds to (or does not correspond to) a normal state in terms of arrhythmia.

As another example, according to one embodiment of the invention, the biosignal analysis model may analyze biosignal data of a subject using a machine learning algorithm (e.g., an artificial neural network) to calculate a score regarding whether the biosignal data corresponds to (or does not correspond to) a specific type of arrhythmia.

Here, according to one embodiment of the invention, the score calculated by the biosignal analysis model may encompass a value for at least one of a probability, a vector, a matrix, and a coordinate regarding correspondence (or non-correspondence) to a normal state or a specific type of arrhythmia.

Meanwhile, the biosignal that may be analyzed by the biosignal analysis model may include a signal regarding an electrocardiogram (ECG), an electromyogram (EMG), an electroencephalogram (EEG), a photoplethysmogram (PPG), a heartbeat, a body temperature, a blood sugar level, a pupil change, a blood pressure level, a blood oxygen content, and the like.

Next, the clustering management unit 220 according to one embodiment of the invention may perform clustering on a plurality of pieces of first-type biosignal data analyzed as corresponding to a first type, among the plurality of pieces of biosignal data.

Here, according to one embodiment of the invention, the first type refers to, in its broadest sense, a type that may be determined by the biosignal analysis model, and may encompass a normal state in terms of arrhythmia, an abnormal state in terms of arrhythmia, and a state corresponding to a specific type of arrhythmia (e.g., atrial premature contraction (APC), atrial fibrillation (AFib), paroxysmal supra ventricular tachycardia (PSVT), and ventricular premature complexes (VPCs)).

Specifically, the clustering management unit 220 according to one embodiment of the invention may cluster a plurality of pieces of first-type biosignal data analyzed as corresponding to the first type into at least one cluster. According to one embodiment of the invention, the biosignal data clustered into the same cluster as the clustering is performed may have features (e.g., patterns, feature points, or waveforms) that are common to each other.

For example, the algorithm that may be used for the biosignal data clustering according to one embodiment of the invention may include k-means, mean shift, Gaussian mixture model (GMM), density-based spatial clustering of applications with noise (DBSCAN), and self-organizing map (SOM). However, it is noted that the clustering algorithm according to the invention is not necessarily limited to those listed above, and may be changed without limitation as long as the objects of the invention may be achieved.

Next, the clustering management unit 220 according to one embodiment of the invention may function to extract at least one piece of sample biosignal data from at least one cluster generated by the clustering. Here, the clustering management unit 220 according to one embodiment of the invention may randomly select and extract at least one piece of sample biosignal data from among a plurality of pieces of biosignal data belonging to a specific cluster.

According to one embodiment of the present invention, an information provision unit (not shown) included in the biosignal analysis system 200 may provide the at least one piece of sample biosignal data extracted as above and analysis result data therefor to the device 300 of an examiner.

That is, according to one embodiment of the invention, the number of the at least one piece of sample biosignal data extracted as above and provided to the examiner (i.e., the number of biosignal data to be examined by the examiner) may be less than the total number of biosignal data belonging to the corresponding cluster at or below a predetermined level, and may be less than the total number of the plurality of pieces of first-type biosignal data determined as corresponding to the first type at or below a predetermined level. Thus, according to the invention, the number of biosignal data to be examined by the examiner may be dramatically reduced.

Further, the clustering management unit 220 according to one embodiment of the invention may reperform the clustering on the plurality of pieces of first-type biosignal data with reference to feedback on whether the analysis result data for the at least one piece of sample biosignal data extracted as above is accurate.

Here, according to one embodiment of the invention, the feedback on whether the analysis result data for the at least one piece of sample biosignal data is accurate may be acquired from the device 300 of the examiner. Specifically, according to one embodiment of the invention, instead of being provided with analysis result data for a large number (e.g., tens of thousands) of biosignal data outputted from the biosignal analysis model, the examiner (e.g., medical personnel such as doctors) may be provided with analysis result data for a small number (e.g., less than dozens) of sample biosignal data extracted through the above clustering, and may examine only the analysis result data for the small number of sample biosignal data and provide feedback on whether the analysis result data is accurate.

For example, when sample biosignal data A, which is analyzed by the biosignal analysis model as corresponding to an atrial premature contraction (APC) type of arrhythmia, is extracted as a sample and provided to the examiner, the examiner may examine the sample biosignal data A and analysis result data therefor and then provide feedback to the effect that the analysis result data is correct (or incorrect).

Next, the clustering management unit 220 according to one embodiment of the invention may reperform the clustering on the plurality of pieces of first-type biosignal data with reference to the above feedback.

Further, the clustering management unit 220 according to one embodiment of the invention may update the clustering algorithm with reference to the feedback.

For example, when feedback is provided to the effect that analysis result data indicating that the sample biosignal data A corresponds to the APC type of arrhythmia is correct, the sample biosignal data A may remain in a clustering target for the APC type (i.e., biosignal data analyzed as corresponding to the APC type and targeted for the clustering).

As another example, when feedback is provided to the effect that analysis result data indicating that sample biosignal data B corresponds to the APC type of arrhythmia is incorrect, the sample biosignal data B may be excluded from the clustering target, and the clustering may be reperformed with the sample biosignal data B being excluded.

As another example, when feedback is provided to the effect that analysis result data indicating that not less than a predetermined percentage (or a predetermined number) of sample biosignal data correspond to the APC type of arrhythmia is incorrect, the clustering may be reperformed with all the biosignal data belonging to the corresponding cluster being excluded.

As another example, when sample biosignal data given feedback that analysis result data is correct and sample biosignal data given feedback that analysis result data is incorrect occur together within a cluster at or above a predetermined level, the clustering algorithm applied to the biosignal data belonging to the corresponding type may be changed.

As discussed above, according to the invention, the clustering may be performed iteratively with reference to the examiner's feedback, so that the accuracy of analysis results for biosignal data clustered as belonging to a specific cluster may be gradually increased, and the accuracy of analysis results for all biosignal data clustered within a specific type encompassing multiple clusters may also be gradually increased.

The clustering management unit 220 according to one embodiment of the invention may dynamically calculate the accuracy of analysis results for at least one piece of sample biosignal data extracted from a specific cluster, on the basis of feedback on the analysis results, and may estimate (or determine) that analysis result data for all biosignal data belonging to the specific cluster is accurate, when the calculated accuracy is at or above a predetermined level.

Further, the clustering management unit 220 according to one embodiment of the invention may dynamically calculate the accuracy of analysis results for a plurality of pieces of first-type biosignal data analyzed as corresponding to the first type, on the basis of feedback on the analysis results, and may estimate (or determine) that analysis result data for all biosignal data analyzed as corresponding to the first type is accurate, when the calculated accuracy is at or above a predetermined level.

Thus, according to the invention, even if the examiner does not examine all analysis result data for all biosignal data outputted from the biosignal analysis model, but examines only analysis result data for a small number of sample biosignal data extracted on the basis of the clustering, a result of the examination (i.e., feedback) may influence the analysis result data for all the biosignal data, thereby increasing the accuracy and reliability of analysis result data that is outputted from the biosignal analysis model and provided to the examiner, while increasing the efficiency of the examination.

Next, the communication unit 230 according to one embodiment of the invention may function to enable data transmission/reception from/to the data acquisition unit 210 and the clustering management unit 220.

Lastly, the control unit 240 according to one embodiment of the invention may function to control data flow among of the data acquisition unit 210, the clustering management unit 220, and the communication unit 230. That is, the control unit 240 according to the invention may control data flow into/out of the biosignal analysis system 200 or data flow among the respective components of the biosignal analysis system 200, such that the data acquisition unit 210, the clustering management unit 220, and the communication unit 230 may carry out their particular functions, respectively.

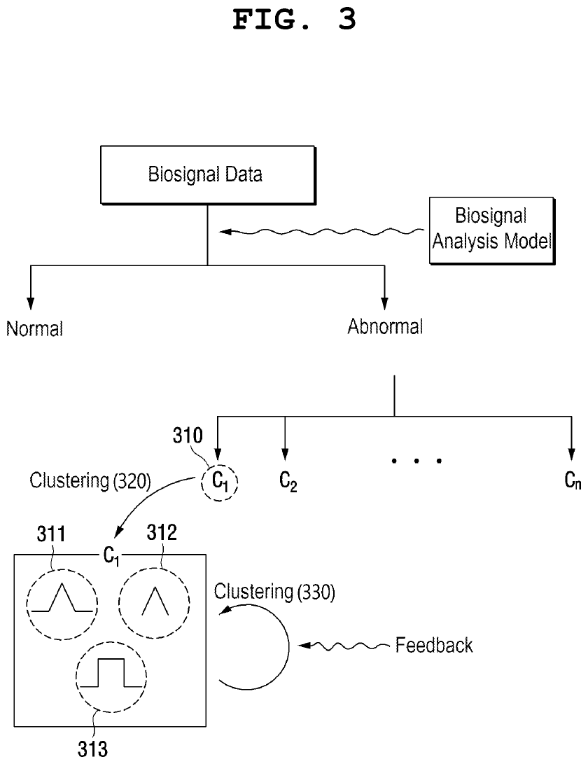

FIG. 3 illustratively shows how to cluster biosignal data according to one embodiment of the invention.

Referring to FIG. 3, it may be assumed that the biosignal data may be determined to be in a normal state or an abnormal state after being analyzed by a biosignal analysis model, and more specifically, may be determined to be a first type (C₁) 310, a second type (C₂), or an n^{th} type (Cₙ), among the abnormal state.

In this case, the biosignal analysis system 200 according to one embodiment of the invention may perform clustering on a plurality of pieces of first-type biosignal data determined by the biosignal analysis model as corresponding to the first type (C₁) 310, thereby clustering the plurality of pieces of first-type biosignal data into at least one cluster (320).

Then, the biosignal analysis system 200 according to one embodiment of the invention may extract at least one piece of sample biosignal data from at least one of a plurality of clusters 311, 312 and 313 generated according to the above clustering.

Then, the biosignal analysis system 200 according to one embodiment of the invention may reperform the clustering on the plurality of pieces of first-type biosignal data 310, with reference to an examiner's feedback on the at least one piece of sample biosignal data extracted as above (330).

Then, when the accuracy of discrimination results for biosignal data belonging to a specific cluster or type is at or above a predetermined level as the clustering is updated as above, the biosignal analysis system 200 according to one embodiment of the invention may estimate (or determine) that the discrimination results for all the biosignal data belonging to the corresponding cluster or type are accurate.

Although the embodiments for analyzing biosignals regarding arrhythmia have been mainly described above, it is noted that the disease that may be analyzed according to the invention is not necessarily limited only to arrhythmia, but the present invention may be utilized for other diseases (e.g., the presence or absence of a disease in another organ such as a brain or a respiratory organ, and the type of the disease) or other technical fields (e.g., the field of instrument abnormality diagnosis or the technique of processing or post-processing data outputted from an analysis model for sensing data acquired from a plurality of sensors) without limitation, as long as the objects of the invention may be achieved.

The embodiments according to the invention as described above may be implemented in the form of program instructions that can be executed by various computer components, and may be stored on a non-transitory computer-readable recording medium. The non-transitory computer-readable recording medium may include program instructions, data files, data structures and the like, separately or in combination. The program instructions stored on the non-transitory computer-readable recording medium may be specially designed and configured for the present invention, or may also be known and available to those skilled in the computer software field. Examples of the non-transitory computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be configured to operate as one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for managing output data of a biosignal analysis model, comprising steps of:
acquiring analysis result data for a plurality of pieces of biosignal data from a biosignal analysis model;
performing clustering on a plurality of pieces of first-type biosignal data analyzed as corresponding to a first type, among the plurality of pieces of biosignal data, on the basis of the analysis result data, and extracting at least one piece of sample biosignal data from at least one cluster generated by the clustering; and
reperforming the clustering with reference to feedback on whether the analysis result data for the at least one piece of sample biosignal data is accurate.

2. The method of claim 1, wherein the biosignal analysis model outputs analysis result data regarding whether the plurality of pieces of biosignal data correspond to arrhythmia, or analysis result data regarding what type of arrhythmia the plurality of pieces of biosignal data correspond to.

3. The method of claim 1, wherein the number of the at least one piece of sample biosignal data is less than the number of the plurality of pieces of first-type biosignal data at or below a predetermined level.

4. The method of claim 1, wherein in the reperforming step, an algorithm used for the clustering is updated.

5. The method of claim 1, wherein accuracy of analysis results for at least one piece of sample biosignal data extracted from a specific cluster is dynamically calculated on the basis of the feedback, and the analysis result data for all biosignal data belonging to the specific cluster is estimated to be accurate when the accuracy is at or above a predetermined level.

6. The method of claim 1, wherein accuracy of analysis results for a plurality of pieces of first-type biosignal data analyzed as corresponding to the first type is dynamically calculated on the basis of the feedback, and the analysis result data for all biosignal data analyzed as corresponding to the first type is estimated to be accurate when the accuracy is at or above a predetermined level.

7. A non-transitory computer-readable recording medium having stored thereon a computer program for executing the method of claim 1.

8. A system for managing output data of a biosignal analysis model, comprising:
a data acquisition unit configured to acquire analysis result data for a plurality of pieces of biosignal data from a biosignal analysis model; and
a clustering management unit configured to perform clustering on a plurality of pieces of first-type biosignal data analyzed as corresponding to a first type, among the plurality of pieces of biosignal data, on the basis of the analysis result data, and extract at least one piece of sample biosignal data from at least one cluster generated by the clustering, and to reperform the clustering with reference to feedback on whether the analysis result data for the at least one piece of sample biosignal data is accurate.

9. The system of claim 8, wherein the biosignal analysis model outputs analysis result data regarding whether the plurality of pieces of biosignal data correspond to arrhythmia, or analysis result data regarding what type of arrhythmia the plurality of pieces of biosignal data correspond to.

10. The system of claim 8, wherein the number of the at least one piece of sample biosignal data is less than the number of the plurality of pieces of first-type biosignal data at or below a predetermined level.

11. The system of claim 8, wherein the clustering management unit is configured to update an algorithm used for the clustering when reperforming the clustering.

12. The system of claim 8, wherein the clustering management unit is configured to dynamically calculate accuracy of analysis results for at least one piece of sample biosignal data extracted from a specific cluster on the basis of the feedback, and estimate that the analysis result data for all biosignal data belonging to the specific cluster is accurate when the accuracy is at or above a predetermined level.

13. The system of claim 8, wherein the clustering management unit is configured to dynamically calculate accuracy of analysis results for a plurality of pieces of first-type biosignal data analyzed as corresponding to the first type on the basis of the feedback, and estimate that the analysis result data for all biosignal data analyzed as corresponding to the first type is accurate when the accuracy is at or above a predetermined level.
